(19) United States
(12) Patent Application Publication
(10) Pub. No.: US 2011/0046438 A1
(43) Pub. Date: Feb. 24, 2011

(54) WIRELESS POWER FEEDING SYSTEM

(75) Inventor: Hiroshi IWAISAKO, Shiojiri-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/914,247

(22) Filed: Oct. 28, 2010

Related U.S. Application Data
(63) Continuation of application No. PCT/JP2009/057974, filed on Apr. 22, 2009.

(30) Foreign Application Priority Data
May 2, 2008 (JP) 2008-120605

(51) Int. Cl. A61B 1/00; H01F 38/00

(52) U.S. Cl. 600/101; 307/104

(57) ABSTRACT

A wireless power feeding system includes a plurality of power transmission antennas, each including a resonance circuit including a power transmission coil and a capacitor located so as to generate a magnetic field in a desired direction, a controller controlling a resonant state of each of the plurality of power transmission antennas, a plurality of driving units applying an AC voltage to the plurality of power transmission antennas to drive each of the plurality of power transmission antennas, and a power supply supplying a voltage to the plurality of driving units. The controller controls a power transmission antenna from which a magnetic field is to be generated to place the power transmission antenna in a resonant state and controls a power transmission antenna from which a magnetic field is not to be generated to place the power transmission antenna in a nonresonant state.

### CROSS REFERENCE TO RELATED APPLICATION

This application is a continuation application of PCT/JP2009/057974 filed on Apr. 22, 2009 and claims benefit of Japanese Application No. 2008-120605 filed in Japan on May 2, 2008, the entire contents of which are incorporated herein by this reference.

### BACKGROUND OF INVENTION

1. Field of the Invention

The present invention relates to a wireless power feeding system which wirelessly supplies electric power from outside of a body to an in-vivo information acquiring device, such as a capsule endoscope, that operates inside of the body.

2. Description of the Related Art

Wireless power feeding system that contactlessly supplies electric power from outside of a body to a certain device such as a capsule endoscope operating in a subject's body has been proposed, such as the one discloses in Japanese Patent Application Laid-Open Publication No. 2004-159456 in which an electric current is passed through primary coils provided in the wireless power feeding system to induce electrical energy in a secondary coil provided in the device.

The configuration of the primary coils provided in the wireless power feeding system in the proposal described in the Japanese Patent Application Laid-Open Publication No. 2004-159456 will be briefly described below with reference to FIGS. 10 and 11.

FIG. 10 illustrates the primary coil configuration of the existing wireless power feeding system. Illustrated in FIG. 10 is the configuration of the wireless power feeding system for capsule endoscope, in which X-, Y-, and Z-axis primary coils are attached to the body of a subject B and electric power is wirelessly supplied to the capsule endoscope which is a small medical device in a body cavity of the subject B.

In FIG. 10, Helmholtz power transmission coil pairs are arranged in a three-dimensional Cartesian coordinate system around the body of a subject B, that is, in the X-, Y-, and Z-axis directions that are orthogonal to each other. Power transmission coils 12a and 12b are placed along the X-axis direction; power transmission coils 13a and 13b are placed along the Y-axis direction; power transmission coils 11a and 11b are placed along the Z-axis direction.

FIG. 11 illustrates a circuit configuration of power transmission coils in the existing wireless power feeding system. When electric power is supplied to a capsule endoscope 100, power transmission coils 11a and 11b, 12a and 12b, and 13a and 13b are connected in pairs in series. The pairs of series-connected power transmission coils 11a and 11b, 12a and 12b, and 13a and 13b are connected to power-transmission-coil resonant capacitors 22, 24 and 26, respectively, in series to form resonance circuits as illustrated in FIG. 11.

The existing wireless power feeding system illustrated in FIG. 11 includes the resonance circuits described above, switching circuits 21, 23 and 25 which supply an AC voltage to the power transmission coils, a power supply unit 15 which supplies electric power to the switching circuits 21, 23 and 35, and switches SW1, SW2 and SW3 which select either supplying or removing electric power to the switching circuits 21, 23 and 25.

In the existing wireless power feeding system described above, a Helmholtz power transmission coil pair that can most efficiently supply electric power, which depends on the location and orientation of the capsule endoscope in the body of the subject B, is selected from among the three Helmholtz power transmission coil pairs and an AC volt is applied to that power transmission coil pair. In this way, electric power can be wirelessly supplied to the capsule endoscope with a high efficiency.

When an AC voltage is applied to power transmission coils to generate a magnetic field, generally a resonance circuit including coils and a capacitor is used in order to increase the efficiency of power supply. As has been described above, in the proposal described in Japanese Patent Application Laid-Open Publication No. 2004-159456, power transmission coils and a resonant capacitor are connected in series in order to increase the efficiency of power supply to each power transmission coil pair and a resonant circuit is used to drive a power transmission antenna.

### SUMMARY OF THE INVENTION

A wireless power feeding system according to one embodiment of the present invention includes: a plurality of power transmission antennas, each including a resonance circuit including a power transmission coil and a capacitor located so as to generate a magnetic field in a desired direction; a controller controlling a resonant state of each of the plurality of power transmission antennas; a plurality of driving units applying an AC voltage to the plurality of power transmission antennas to drive each of the plurality of power transmission antennas; and a power supply unit supplying a voltage to the driving units; wherein the controller controls, among the plurality of power transmission antennas, a power transmission antenna from which a magnetic field is to be generated to place the power transmission antenna in a resonant state and controls, among the plurality of power transmission antennas, a power transmission antenna from which a magnetic field is not to be generated to place the power transmission antenna in a nonresonant state.

### BRIEF DESCRIPTION OF THE DRAWINGS

FIG. 1 is a schematic diagram illustrating a configuration of a wireless power feeding system according to a first embodiment of the present invention;

FIG. 2 is a schematic front view illustrating power transmission coils 43, 53 and 63 located around the body of a subject along three axes, viewed from the front of the subject;

FIG. 3 is a schematic cross-sectional view illustrating the power transmission coils 43, 53 and 63 located around the body of the subject along the three axes, viewed from above the subject;

FIG. 4 is a schematic diagram illustrating a configuration of a wireless power feeding system according to a second embodiment of the present invention;

FIG. 5 is a schematic diagram illustrating a configuration of a wireless power feeding system according to a third embodiment of the present invention;

FIG. 6 is a schematic diagram illustrating a configuration of a wireless power feeding system according to a fourth embodiment of the present invention;

FIG. 7 is a schematic diagram illustrating a configuration of a wireless power feeding system according to a fifth embodiment of the present invention;

FIG. 8 is a schematic diagram illustrating a configuration of a wireless power feeding system according to a sixth embodiment of the present invention;

FIG. 9 is a schematic diagram illustrating a variation of the wireless power feeding system according to the sixth embodiment of the present invention;

FIG. 10 is a diagram illustrating a primary coil configuration in an existing wireless power feeding system; and

FIG. 11 is a circuit diagram of primary coils in the existing wireless power feeding system.

### DETAILED DESCRIPTION OF THE PREFERRED EMBODIMENT(S)

Embodiments of the present invention will be described with reference to drawings.

### First Embodiment

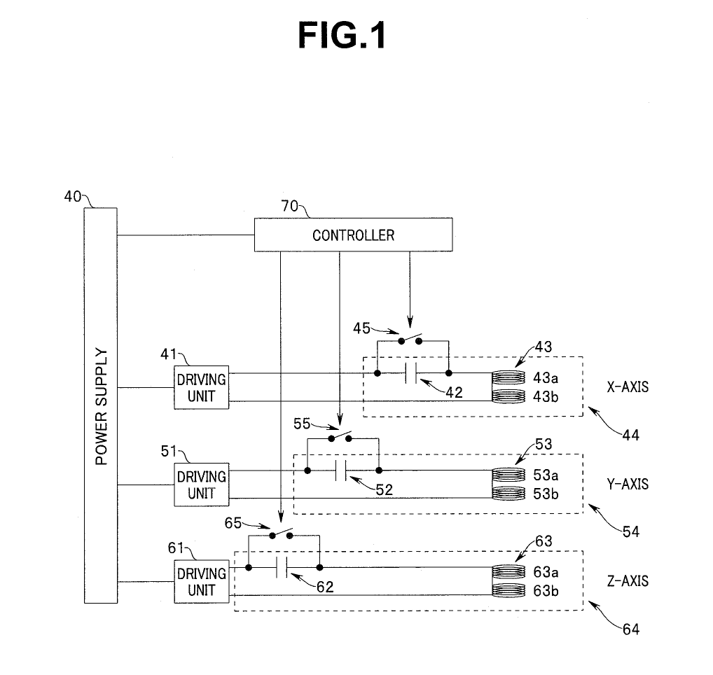

A configuration of a wireless power feeding system will be described first with reference to FIG. 1. FIG. 1 is a schematic diagram illustrating a configuration of a wireless power feeding system according to a first embodiment of the present invention.

The wireless power feeding system in FIG. 1 mainly includes three power transmission antennas 44, 54 and 64, driving units 41, 51 and 61 which drive the power transmission antennas 44, 54 and 64, a controller 70 which performs control to allow an electric current to flow through capacitors 42, 52, 62 or to flow without passing through the capacitors 42, 52, 62, and a power supply 40 which is electrically connected to the driving units 41, 51 and 61 and the controller 70 and supplies electric power to the driving units 41, 51 and 61 and the controller 70.

The power supply 40 is an alternating current (AC) power supply, an AC-to-direct-current (DC) conversion power supply, or a direct-current (DC) power supply, or the like. The driving units 41, 51 and 61 are electrically connected to the power transmission antennas 44, 54 and 64, respectively, and apply a voltage outputted from the power supply 40 to the power transmission antennas 44, 54 and 64, respectively.

The power transmission antenna 44 is formed by a series resonance circuit in which the capacitor 42 and a power transmission coil 43 for X-axis drive are connected in series. The power transmission coil 43 includes coils 43a and 43b connected in a Helmholtz arrangement.

A switch 45 is connected to the ends of the capacitor 42 for controlling a resonant state of the power transmission antenna 44. Specifically, the switch 45 is connected with the capacitor 42 in parallel and is capable of turning on and off a current flow across the capacitor 42. When the switch 45 is turned off (open), a current flows to the power transmission coil 43 through the capacitor 42; when the switch 45 is turned on (in conduction), no current flows through the capacitor 42 but a current flows to the power transmission coil 43 through the switch 45.

Like the power transmission antenna 44, the power transmission antenna 54 is formed by a series resonance circuit in which the capacitor 52 and a power transmission coil 53 for Y-axis drive are connected in series. The power transmission coil 53 includes coils 53a and 53b connected in a Helmholtz arrangement.

A switch 55 is connected to the ends of the capacitor 52 for controlling a resonant state of the power transmission antenna 54. Specifically, the switch 55 is connected with the capacitor 52 in parallel and is capable of turning on and off a current flow across the capacitor 52. When the switch 55 is turned off (open), a current flows to the power transmission coil through the capacitor 52; when the switch 55 is turned on (in conduction), no current flows through the capacitor 52 but a current flows to the power transmission coil 53 through the switch 55.

Like the power transmission antennas 44 and 54, the power transmission antenna 64 is formed by a series resonance circuit in which the capacitor 62 and a power transmission coil 63 for Z-axis drive are connected in series. The power transmission coil 63 includes coils 63a and 63b connected in a Helmholtz arrangement.

A switch 65 is connected to the ends of the capacitor 62 for controlling a resonant state of the power transmission antenna 64. Specifically, the switch 65 is connected with the capacitor 62 in parallel and is capable of turning on and off a current flow across the capacitor 62. When the switch 65 is turned off (open), a current flows to the power transmission coil 63 through the capacitor 62; when the switch 65 is turned on (in conduction), no current flows through the capacitor 62 but a current flows to the power transmission coil 63 through the switch 65.

Each of the switches 45, 55 and 65 is turned on and off according to a control signal from the controller 70. The switches 45, 55 and 65 are not limited to switches with contacts and semiconductor switches; they may be any switches that are capable of switching between blocking and maintaining a current path.

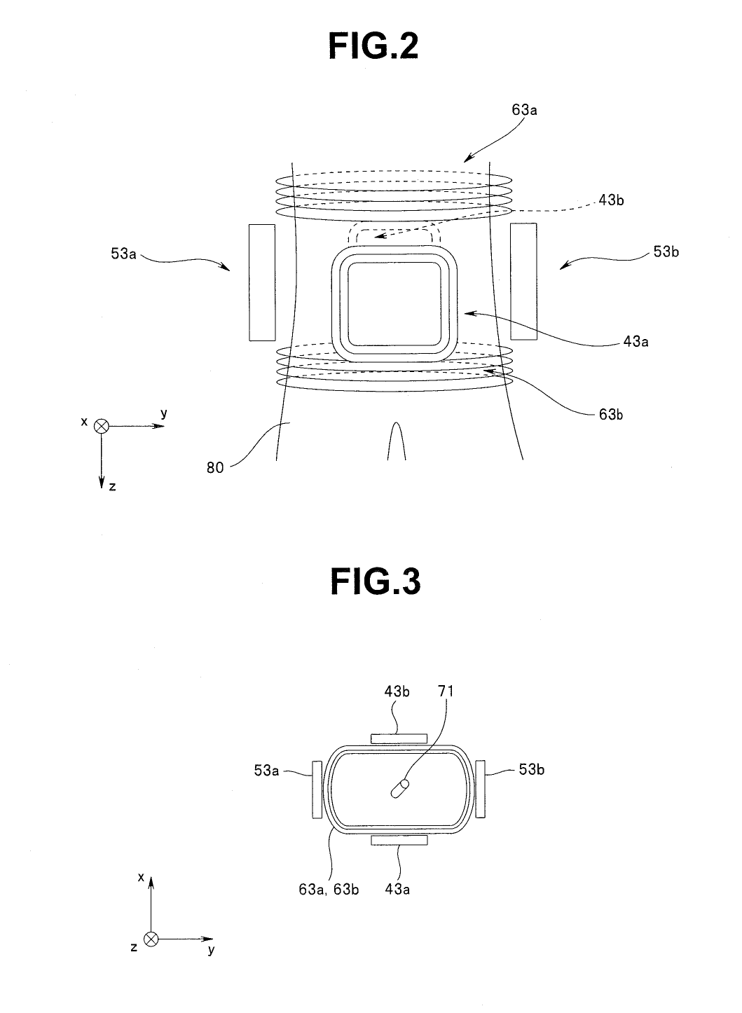

The power transmission coil 43 for X-axis drive, the power transmission coil 53 for Y-axis drive, and the power transmission coil 63 for Z-axis drive are located in a substantial three-dimensional Cartesian coordinate system around the body of a subject as illustrated in FIGS. 2 and 3. FIG. 2 is a schematic front view illustrating the power transmission coils 43, 53 and 63 located around the body of the subject along the three axes, viewed from the front of the subject. FIG. 3 is a schematic cross-sectional view illustrating the power transmission coils 43, 53 and 63 located around the body of the subject along the three axes, viewed from above the subject.

Applying a high-frequency voltage to one of the X-, Y-, and Z-axis power transmission coils can cause the power transmission coil to generate an AC magnetic field to wirelessly supply electric power to a capsule endoscope 71, which is a in-vivo information acquiring device retained inside the body of a subject 80.

An operation of the wireless power feeding system configured as described above will be described below. An operation to apply an AC voltage only to the X-axis power transmission coil among the X-, Y-, and Z-axis power transmission coils illustrated in FIGS. 2 and 3 to generate a magnetic field for the X-axis will be described first.

First, electric power is supplied from the power supply 40 to the driving unit 41. In the driving unit 41, the supplied electric power is converted to high-frequency power with the same frequency as a resonance frequency of the series resonance circuit made up of the capacitor 42 and the power transmission coil 43. The high-frequency power is applied by the driving unit 41 to the power transmission antenna 44 formed by the capacitor 42 and the power transmission coil 43 connected in series.

Since a magnetic field does not need to be generated at the power transmission coils 53 and 63, an AC current (high-frequency voltage) is not generated in the driving unit 51 and 61 connected to the power transmission coils 53 and 63.

The controller 70 sends out a control signal to turn off the switch 45 attached to the X-axis power transmission antenna 44. When the switch 45 is turned off, the power transmission antenna 44 can form a resonance circuit. Consequently, a magnetic field is generated at the power transmission coil 43 by the applied high-frequency power.

At the same time, the controller sends a control signal to turn on the switch 55 attached to the Y-axis power transmission antenna 54 and the switch 65 attached to the Z-axis power transmission antenna 64. When the switches 55 and 65 are turned on by the control, the power transmission antennas 54 and 64 become unable to form resonance circuits.

Accordingly, even if the Y-axis power transmission coil 53 and/or the Z-axis power transmission coil 63 is exposed to the magnetic field generated from the X-axis power transmission coil 43, generation of an induced electromotive force by the magnetic field can be sufficiently inhibited. Therefore a magnetic field can be generated only from the X-axis power transmission coil 43 with a desired intensity in a desired orientation.

An operation to apply an AC voltage only to the Y-axis to generate a magnetic field for the Y-axis will be described next. In this case, the X-axis components for generating the magnetic field for the X-axis in the above description can simply be replaced with the Y-axis components equivalent to those components. Specifically, the controller 70 sends out a control signal to turn off the switch 55 attached to the Y-axis power transmission antenna 54. At the same time, the controller 70 sends out a control signal to turn on the switch 45 attached to the X-axis power transmission antenna 44 and the switch 65 attached to the Z-axis power transmission antenna 64. Electric power supplied from the power supply 40 is converted to an AC voltage (high-frequency voltage) by the driving unit 51 and the high-frequency voltage is applied only to the Y-axis power transmission antenna 54.

With the operation described above, even if the X-axis power transmission coil 43 and/or the Z-axis power transmission coil 63 is exposed to the magnetic field generated from the Y-axis power transmission coil 53, generation of an induced electromotive force by the magnetic field can be sufficiently inhibited. Accordingly, a magnetic field can be generated only from the Y-axis power transmission coil 53 with a desired intensity in a desired orientation.

When an AC voltage is to be applied only to the Z-axis to generate a magnetic field for the Y-axis, an operation and control similar to those for the X and Y axes are performed. Specifically, the controller 70 sends out a control signal to turn off the switch 65 attached to the Z-axis power transmission antenna 64. At the same time, the controller 70 sends out a control signal to turn on the switch 45 attached to the X-axis power transmission antenna 44 and the switch 55 attached to the Y-axis power transmission antenna 54. Electric power supplied from the power supply 40 is converted to an AC voltage (high-frequency power) by the driving unit 61 and the high-frequency power is applied only to the Z-axis power transmission antenna 64.

With the operation described above, even if the X-axis power transmission coil 43 and/or the Y-axis power transmission coil 53 is exposed to the magnetic field generated from the Z-axis power transmission coil 63, generation of an induced electromotive force by the magnetic field can be sufficiently prevented. Accordingly, a magnetic field can be generated only from only the Z-axis power transmission coil 63 with a desired intensity in a desired orientation.

In this way, since the switches 45, 55 and 65 are provided at the power transmission antennas 44, 54 and 64 of the three axes and can be turned on and off by the controller 70 to place only a power transmission antenna of an axis from which a magnetic filed is to be generated in a resonant state and to place the other power transmission antennas of the other two axes in a nonresonant state in the wireless power feeding system of the present embodiment, the magnitude of an electric current to pass through each power transmission coil can be stably controlled and the intensity and orientation of the magnetic field generated from the power transmission coil can be properly controlled. Accordingly, electric power can be efficiently supplied to the in-vivo information acquiring device. Furthermore, an induced electromotive force can be inhibited from being generated between power transmission coils and therefore useless electric power is eliminated and energy savings can be achieved.

### Second Embodiment

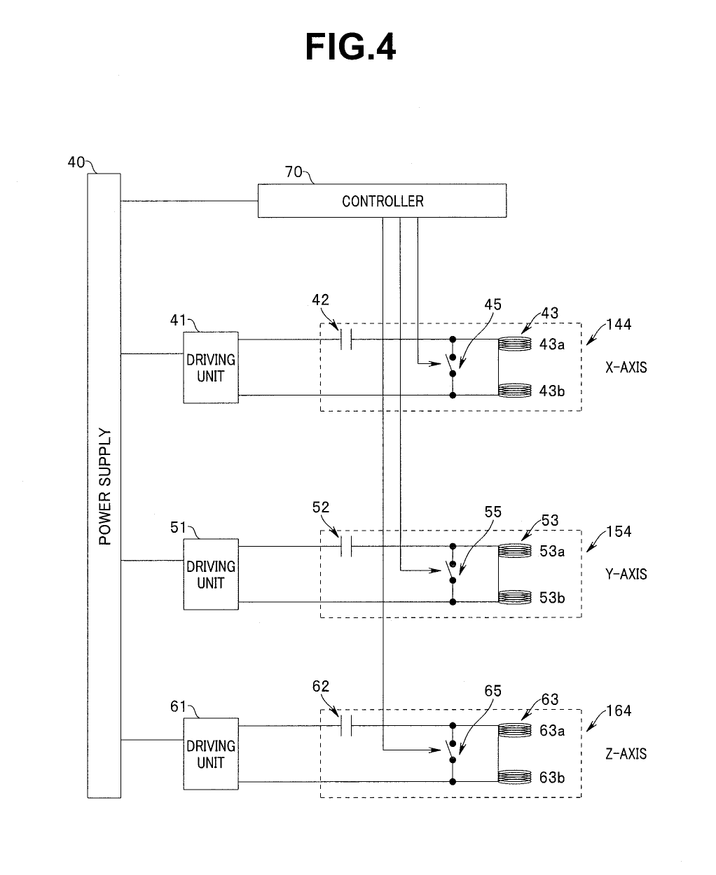

A wireless power feeding system according to a second embodiment of the present invention will be described with reference to FIG. 4 in detail. FIG. 4 is a schematic diagram illustrating a configuration of the wireless power feeding system according to the second embodiment of the present invention.

The configuration of the wireless power feeding system of the present embodiment is the same as that of the wireless power feeding system of the first embodiment described with reference to FIG. 1 with the only difference being the circuit configuration of power transmission antennas 144, 154 and 164 corresponding to X-, Y- and Z-axes, respectively. Therefore, only the circuit configuration of the power transmission antennas 144, 154 and 164 will be described here and the same components as those of the first embodiments are given the same reference symbols and description of the same components will be omitted.

The power transmission antennas 144, 154 and 164 of the X-, Y-, and Z-axes have the same circuit configuration. Therefore only the X-axis power transmission antenna 144 will be described here and description of the Y- and Z-axis power transmission antennas 154 and 164 will be omitted.

In the first embodiment, the switch 45 is connected to the ends of the capacitor 42 in the power transmission antenna 44 including the series resonance circuit in which the capacitor 42 and the power transmission coil 43 are connected in series. The second embodiment differs from the first embodiment in that a switch 45 is connected to the ends of a power transmission coil 43 in a power transmission antenna 144 including a series resonance circuit in which a capacitor 42 and a power transmission coil 43 are connected in series.

The switch 45 turns on and off the connection between the ends of the power transmission coil 43 according to a control signal sent from a controller 70. Similarly, switches 55 and 65 are connected to the ends of a Y-axis power transmission coil 53 and a Z-axis power transmission coil 63, respectively, as in the power transmission antenna 144.

Specifically, when a magnetic field is to be generated along the X-axis, the switch 45 is turned off. At the same time, the switches 55 and 65 for the Y- and Z-axes are turned on. By controlling and turning on and off the switches 45, 55 and 65 in this way, the X-axis power transmission antenna 144 is placed in a resonant state and the Y-axis power transmission antenna 154 and the Z-axis power transmission antenna 164 are placed in a nonresonant state. Accordingly, a magnetic field can be generated only from the X-axis power transmission antenna with a desired intensity in a desired orientation.

Control of turning on and off of the switches to generate a magnetic field for the Y axis and a magnetic field for the Z axis is similar to the control for the X-axis and can be achieved simply by replacing the components used for the X-axis with the components for the Y- and Z-axes, as in the first embodiment. Therefore description of operations for the Y- and Z-axes will be omitted.

In this way, since the switches 45, 55 and 65 are provided at the power transmission antennas 144, 154 and 164 of the three axes and can be turned on and off by the controller 70 to place only a power transmission antenna of an axis from which a magnetic filed is to be generated in a resonant state and to place the other power transmission antennas of the other two axes in a nonresonant state in the present embodiment, the magnitude of an electric current to pass through each power transmission coil can be stably controlled and the intensity and orientation of the magnetic field generated from the power transmission coil can be properly controlled. Accordingly, electric power can be efficiently supplied to the in-vivo information acquiring device. Furthermore, an induced electromotive force can be inhibited from being generated between power transmission coils and therefore useless electric power is eliminated and energy savings can be achieved.

### Third Embodiment

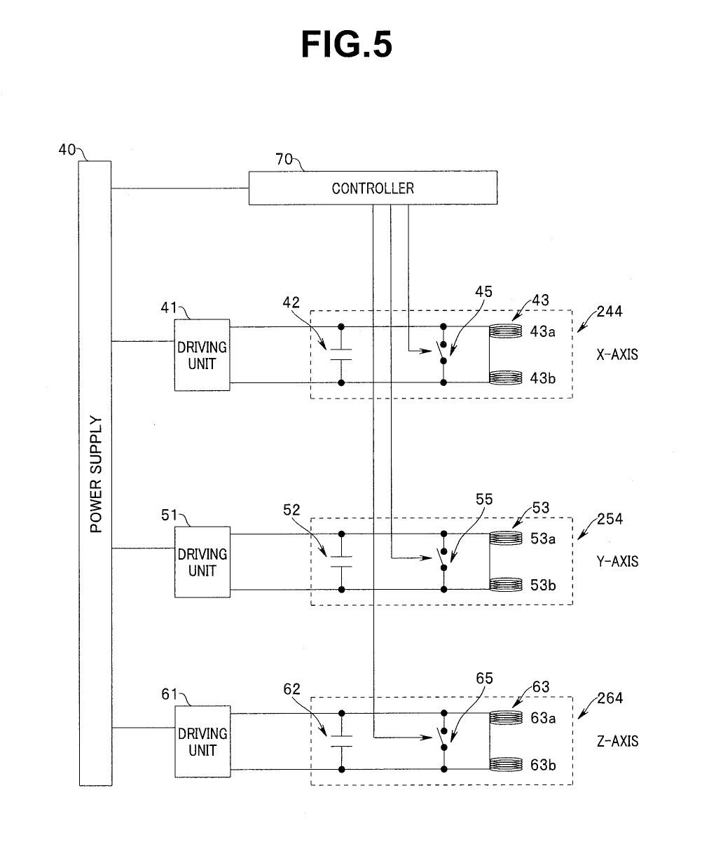

A wireless power feeding system according to a third embodiment of the present invention will be described below with reference to FIG. 5 in detail. FIG. 5 is a schematic diagram illustrating a configuration of the wireless power feeding system according to the third embodiment of the present invention.

The configuration of the wireless power feeding system of the present embodiment is the same as the wireless power feeding system of the first embodiment described with reference to FIG. 1 with the only difference being the circuit configuration of power transmission antennas 244, 254 and 264 corresponding to the X-, Y- and Z axes, respectively. Therefore, only the circuit configuration of the power transmission antennas 244, 254 and 264 will be described here and the same components as those of the first embodiment are given the same reference symbols and description of the same components will be omitted.

The X-, Y- and Z-axis power transmission antennas 244, 254 and 264 have the same circuit configuration. Therefore only the X-axis power transmission antenna 244 will be described here and description of the Y- and Z-axis power transmission antennas 254 and 264 will be omitted.

In the first embodiment, the switch 45 is connected to the ends of the capacitor 42 in the power transmission antenna 44 including a series resonance circuit in which the capacitor 42 and the power transmission coil 43 are connected in series. The present embodiment differs from the first embodiment in that a switch 45 is connected to the ends of a capacitor 42, that is, to the ends of a power transmission coil 43, in a power transmission antenna 244 including a parallel resonance circuit in which the capacitor 42 and the power transmission coil 43 are connected in parallel.

The switch 45 turns on and off the connection between the ends of power transmission coil 43 according to a control signal sent from a controller 70. Switches 55 and 65 are connected to the ends of a Y-axis power transmission coil 53 and a Z-axis power transmission coil 63, respectively, as in the power transmission antenna 244.

Specifically, when a magnetic field is to be generated along the X-axis, the switch 45 is turned off. At the same time, the switches 55 and 65 for the Y- and Z-axes are turned on. By controlling and turning on and off the switches 45, 55 and 65 in this way, the X-axis power transmission antenna 244 is placed in a resonant state and the Y-axis power transmission antenna 254 and the Z-axis power transmission antenna 264 are placed in a nonresonant state. Accordingly, a magnetic field can be generated only from the X-axis power transmission antenna with a desired intensity in a desired orientation.

Control of turning on and off of the switches to generate a magnetic field for the Y axis and a magnetic field for the Z axis is similar to the control for the X-axis and can be achieved simply by replacing the components used for the X-axis with the components for the Y- and Z-axes, as in the first embodiment. Therefore description of operations for the Y- and Z-axes will be omitted.

In this way, since the switches 45, 55 and 65 are provided at the power transmission antennas 244, 254 and 264 of the three axes and can be turned on and off by the controller 70 to place only a power transmission antenna of an axis from which a magnetic filed is to be generated in a resonant state and to place the other power transmission antennas of the other two axes in a nonresonant state in the present embodiment, the magnitude of an electric current to pass through each power transmission coil can be stably controlled and the intensity and orientation of the magnetic field generated from the power transmission coil can be properly controlled. Accordingly, electric power can be efficiently supplied to the in-vivo information acquiring device. Furthermore, an induced electromotive force can be inhibited from being generated between power transmission coils and therefore useless electric power is eliminated and energy savings can be achieved.

### Fourth Embodiment

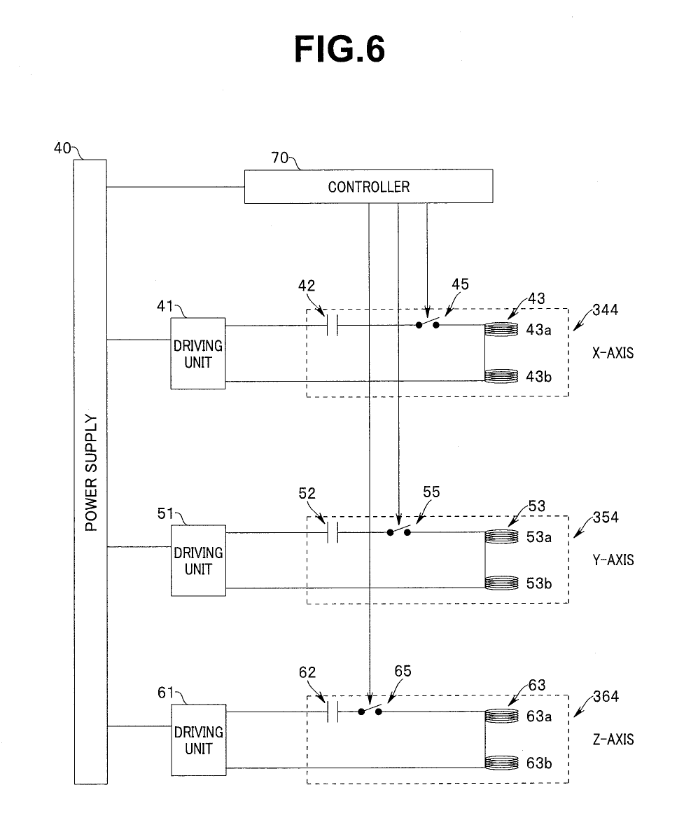

A wireless power feeding system according to a fourth embodiment of the present invention will be described with reference to FIG. 6 in detail. FIG. 6 is a schematic diagram illustrating a configuration of the wireless power feeding system according to the fourth embodiment of the present invention.

The configuration of the wireless power feeding system of the present embodiment is the same as the wireless power feeding system of the first embodiment described with reference to FIG. 1 with the only difference being the circuit configuration of power transmission antennas 344, 354 and 364 corresponding to the X-, Y- and Z axes, respectively. Therefore, only the circuit configuration of the power transmission antennas 344, 354 and 364 will be described here and the same components as those of the first embodiment are given the same reference symbols and description of the same components will be omitted.

The X-, Y- and Z-axis power transmission antennas 344, 354 and 364 have the same circuit configuration. Therefore only the X-axis power transmission antenna 344 will be described here and description of the Y- and Z-axis power transmission antennas 354 and 364 will be omitted.

In the first embodiment, the switch 45 is connected to the ends of the capacitor 42 in the power transmission antenna 44 including a series resonance circuit in which the capacitor 42 and the power transmission coil 43 are connected in series. The present embodiment differs from the first embodiment in that a switch 45 is connected between a capacitor 42 and a power transmission coil 43 in a power transmission antenna 344 including a series resonance circuit in which the capacitor 42 and the power transmission coil 43 are connected in series.

The switch 45 turns on and off the connection between the capacitor 42 and the power transmission coil 43 according to a control signal sent from a controller 70. Switches 55 and 65 are connected between a capacitor 52 and a power transmission coil 53 of the Y-axis and between a capacitor 62 and a power transmission coil 63 of the Z-axis, respectively, as in the power transmission antenna 344.

Specifically, when a magnetic field is to be generated along the X-axis, the switch 45 is turned on. At the same time, the switches 55 and 65 for the Y- and Z-axes are turned off. By controlling and turning on and off the switches 45, 55 and 65 in this way, the X-axis power transmission antenna 344 is placed in a resonant state and the Y-axis power transmission antenna 354 and the Z-axis power transmission antenna 364 are placed in a nonresonant state. Accordingly, a magnetic field can be generated only from the X-axis power transmission antenna with a desired intensity in a desired orientation.

Control of turning on and off of the switches to generate a magnetic field for the Y axis and a magnetic field for the Z axis is similar to the control for the X-axis and can be achieved simply by replacing the components used for the X-axis with the components for the Y- and Z-axes, as in the first embodiment. Therefore description of operations for the Y- and Z-axes will be omitted.

In this way, since the switches 45, 55 and 65 are provided at the power transmission antennas 344, 354 and 364 of the three axes and can be turned on and off by the controller 70 to place only a power transmission antenna of an axis from which a magnetic filed is to be generated in a resonant state and to place the other power transmission antennas of the other two axes in a nonresonant state in the present embodiment, the magnitude of an electric current to pass through each power transmission coil can be stably controlled and the intensity and orientation of the magnetic field generated from the power transmission coil can be properly controlled. Accordingly, electric power can be efficiently supplied to the in-vivo information acquiring device. Furthermore, an induced electromotive force can be inhibited from being generated between power transmission coils and therefore useless electric power is eliminated and energy savings can be achieved.

### Fifth Embodiment

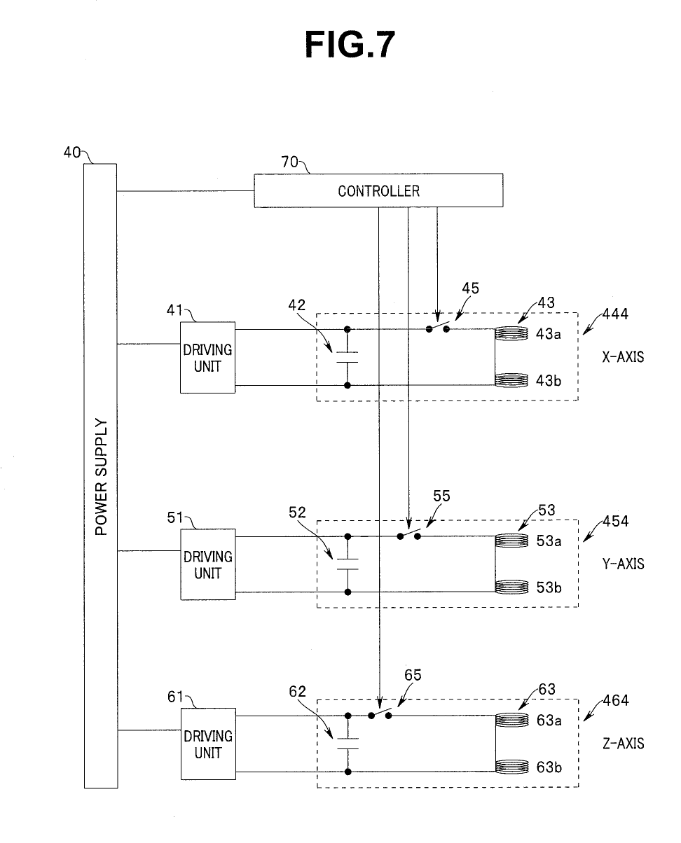

A wireless power feeding system according to a fifth embodiment of the present invention will be described with reference to FIG. 7 in detail. FIG. 7 is a schematic diagram illustrating a configuration of the wireless power feeding system according to the fifth embodiment of the present invention.

The configuration of the wireless power feeding system of the present embodiment is the same as the wireless power feeding system of the first embodiment described with reference to FIG. 1 with the only difference being the circuit configuration of power transmission antennas 444, 454 and 464 corresponding to the X-, Y- and Z axes, respectively. Therefore, only the circuit configuration of the power transmission antennas 444, 454 and 464 will be described here and the same components as those of the first embodiment are given the same reference symbols and description of the same components will be omitted.

The X-, Y- and Z-axis power transmission antennas 444, 454 and 464 have the same circuit configuration. Therefore only the X-axis power transmission antenna 444 will be described here and description of the Y- and Z-axis power transmission antennas 454 and 464 will be omitted.

In the first embodiment, the switch 45 is connected to the ends of the capacitor 42 in the power transmission antenna 44 including a series resonance circuit in which the capacitor 42 and the power transmission coil 43 are connected in series. The present embodiment differs from the first embodiment in that a switch 45 is connected to at least one of two connection points between a capacitor 42 and a power transmission coil 43 in a power transmission antenna 444 including a parallel resonance circuit in which the capacitor 42 and the power transmission coil 43 are connected in parallel.

The switch 45 turns on and off the connection between the capacitor 42 and the power transmission coil 43 according to a control signal sent from the controller 70. As in the power transmission antenna 344, a switch 55 is connected to at least one of two connection points between a capacitor 52 and a power transmission coil 53 of the Y axis and a switch 65 is connected to at least one of two connection points between a capacitor 62 and a power transmission coil 63 of the Z-axis.

Specifically, when a magnetic field is to be generated along the X-axis, the switch 45 is turned on. At the same time, the switches 55 and 65 for the Y- and Z-axes are turned off. By controlling and turning on and off the switches 45, 55 and 65 in this way, the X-axis power transmission antenna 444 is placed in a resonant state and the Y-axis power transmission antenna 454 and the Z-axis power transmission antenna 464 are placed in a nonresonant state. Accordingly, a magnetic field can be generated only from the X-axis power transmission antenna with a desired intensity in a desired orientation.

Control of turning on and off of the switches to generate a magnetic field for the Y axis and a magnetic field for the Z axis is similar to the control for the X-axis and can be achieved simply by replacing the components used for the X-axis with the components for the Y- and Z-axes. Therefore description of operations for the Y- and Z-axes will be omitted.

In this way, since the switches 45, 55 and 65 are provided at the power transmission antennas 444, 454 and 464 of the three axes and can be turned on and off by the controller 70 to place only a power transmission antenna of an axis from which a magnetic filed is to be generated in a resonant state and to place the other power transmission antennas of the other two axes in a nonresonant state in the present embodiment, the magnitude of an electric current to pass through each power transmission coil can be stably controlled and the intensity and orientation of the magnetic field generated from the power transmission coil can be properly controlled. Accordingly, electric power can be efficiently supplied to the in-vivo information acquiring device. Furthermore, an induced electromotive force can be inhibited from being generated between power transmission coils and therefore useless electric power is eliminated and energy savings can be achieved.

### Sixth Embodiment

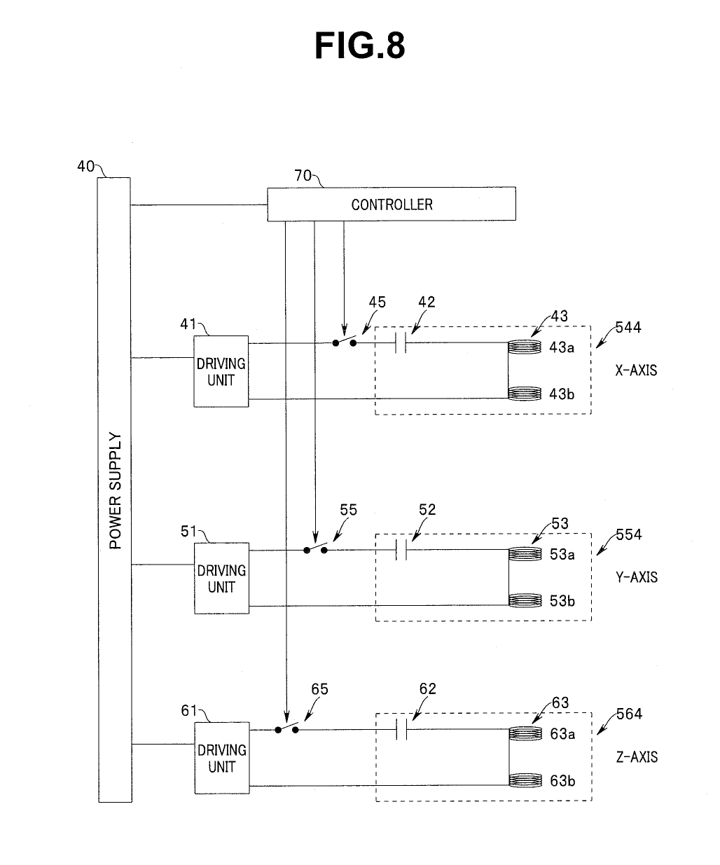

A wireless power feeding system according to a sixth embodiment of the present invention will be described with reference to FIG. 8 in detail. FIG. 8 is a schematic diagram illustrating a configuration of the wireless power feeding system according to the sixth embodiment of the present invention.

The configuration of the wireless power feeding system of the present embodiment is the same as the wireless power feeding system of the first embodiment described with reference to FIG. 1 with the only difference being the circuit configuration of power transmission antennas 544, 554 and 564 corresponding to the X-, Y- and Z axes, respectively. Therefore, only the circuit configuration of the power transmission antennas 544, 554 and 564 will be described here and the same components as those of the first embodiment are given the same reference symbols and description of the same components will be omitted.

The X-, Y- and Z-axis power transmission antennas 544, 554 and 564 have the same circuit configuration. Therefore only the X-axis power transmission antenna 544 will be described here and description of the Y- and Z-axis power transmission antennas 554 and 564 will be omitted.

In the first embodiment, the switch 45 is connected to the ends of the capacitor 42 in the power transmission antenna 44 including a series resonance circuit in which the capacitor 42 and the power transmission coil 43 are connected in series. The present embodiment differs from the first embodiment in that a switch 45 is connected between a driving unit 41 and a capacitor 42 in a power transmission antenna 544 including a series resonance circuit in which the capacitor 42 and the power transmission coil 43 are connected in series.

The switch 45 turns on and off the connection between the driving unit 41 and the capacitor 42 according to a control signal sent from the controller 70. As in the power transmission antenna 544, a switch 55 is connected between a driving unit 51 and a capacitor 52 of the Y-axis and a switch 65 is connected between a driving unit 61 and a capacitor 62 of the Z-axis.

Specifically, when a magnetic field is to be generated along the X-axis, the switch 45 is turned on. At the same time, the switches 55 and 65 for the Y- and Z-axes are turned off. By controlling and turning on and off the switches 45, 55 and 65 in this way, the X-axis power transmission antenna 544 is placed in a resonant state and the Y-axis power transmission antenna 554 and the Z-axis power transmission antenna 564 are placed in a nonresonant state. Accordingly, a magnetic field can be generated only from the X-axis power transmission antenna with a desired intensity in a desired orientation.

Control of turning on and off of the switches to generate a magnetic field for the Y axis and a magnetic field for the Z axis is similar to the control for the X-axis and can be achieved simply by replacing the components used for the X-axis with the components for the Y- and Z-axes. Therefore description of operations for the Y- and Z-axes will be omitted.

In this way, since the switches 45, 55 and 65 are provided at the power transmission antennas 544, 554 and 564 of the three axes and can be turned on and off by the controller 70 to place only a power transmission antenna of an axis from which a magnetic filed is to be generated in a resonant state and to place the other power transmission antennas of the other two axes in a nonresonant state in the present embodiment, the magnitude of an electric current to pass through each power transmission coil can be stably controlled and the intensity and orientation of the magnetic field generated from the power transmission coil can be properly controlled. Accordingly, electric power can be efficiently supplied to the in-vivo information acquiring device. Furthermore, an induced electromotive force can be inhibited from being generated between power transmission coils and therefore useless electric power is eliminated and energy savings can be achieved.

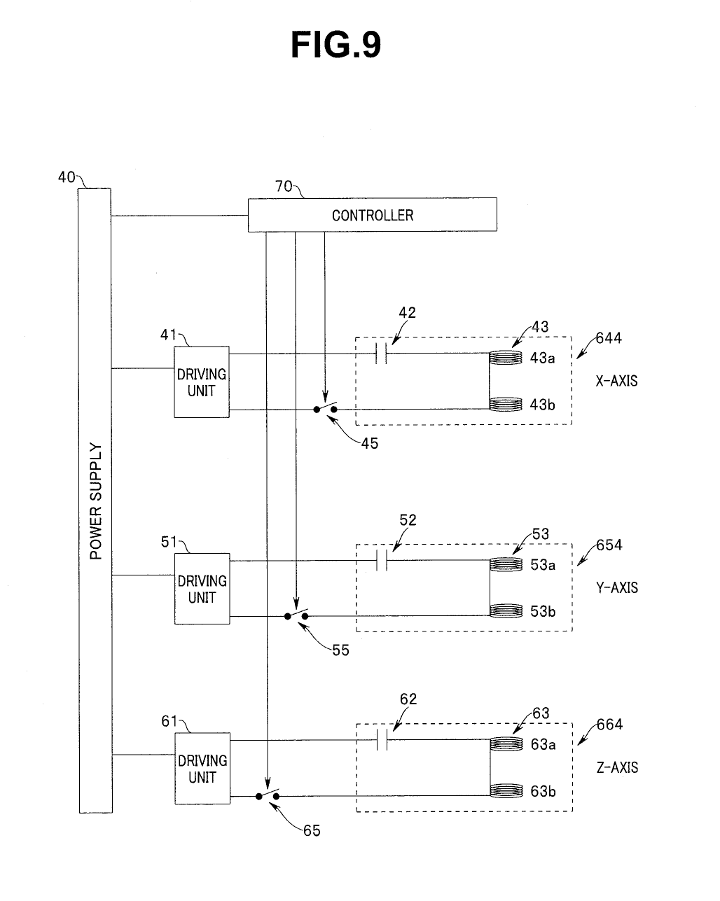

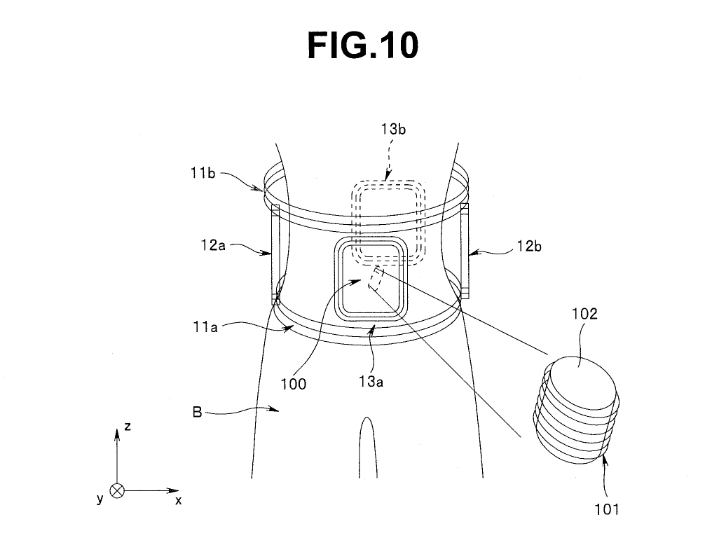

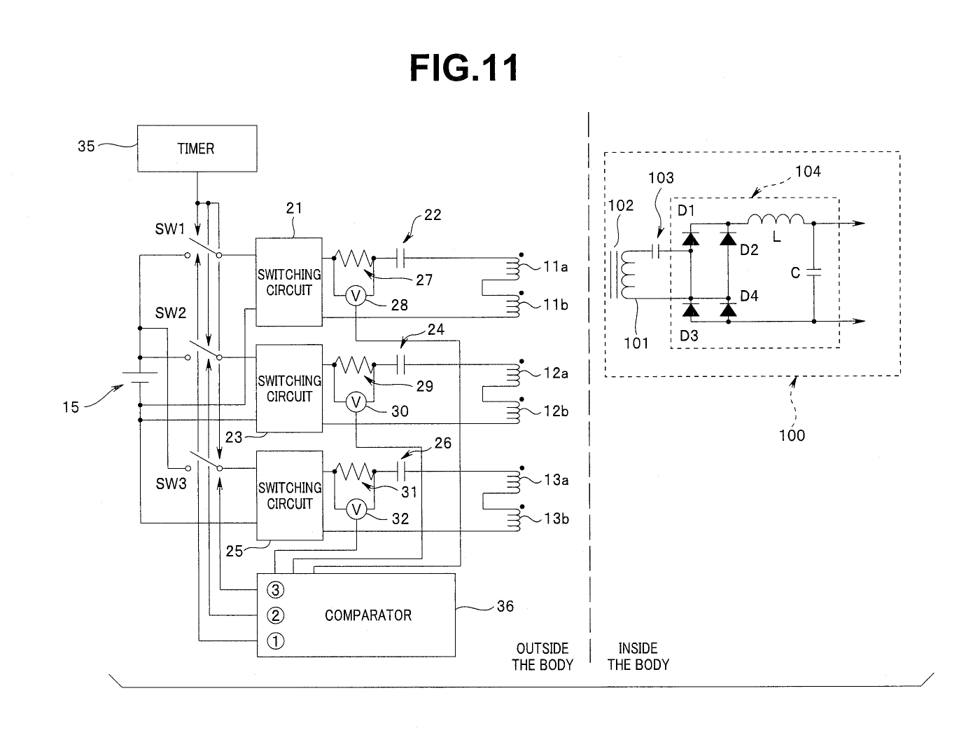

While each switch 45, 55, 65 is provided between the driving unit 41, 51, 61, and the capacitor 42, 52, 62 in the present embodiment, the circuit configuration of the power transmission antennas may be modified as illustrated in FIG. 9 because it is essential only that switching control of (on and off of) the electrical connection between the driving unit 41, 51, 61 and the power transmission antenna 544, 554, 564 can be accomplished with a switch 45, 55, 65. FIG. 9 is a schematic diagram illustrating a variation of the wireless power feeding system according to the sixth embodiment of the present invention.

As illustrated in FIG. 9, the switch 45, 55, 65 may be provided between the driving unit 41, 51, 61 and the power transmission coil 43, 53, 63.

As has been described above, according to any of the embodiments described above, there can be provided a wireless power feeding system capable of efficiently supplying electric power by stably controlling the magnitude of an electric current passing through a power transmission coil and appropriately controlling the intensity and orientation of a magnetic field generated by the power transmission coil.

While the six embodiments have been described with respect to power transmission antennas or three axes, X, Y and Z, the present invention is applicable to power transmission antennas of more than one axis.

While wireless power feeding systems of the present invention have been described with respect to a capsule endoscope as an example of the in-vivo information acquiring device, the present invention is not limited to the embodiments described above. Various changes and modifications can be made to the embodiments without departing from the spirit of the present invention.

For example, the present invention is also applicable to a physiological sensor or a medical device as an in-vivo information acquiring system.

It will be understood that the wireless power feeding system of the present invention is applicable to a wide variety of apparatuses that wirelessly supply electric power, in addition to in-vivo information acquiring devices mentioned above.

What is claimed is:

1. A wireless power feeding system comprising:
a plurality of power transmission antennas, each comprising a resonance circuit including a power transmission coil and a capacitor located so as to generate a magnetic field in a desired direction;
a controller controlling a resonant state of each of the plurality of power transmission antennas;
a plurality of driving units applying an AC voltage to the plurality of power transmission antennas to drive each of the plurality of power transmission antennas; and
a power supply unit supplying a voltage to the driving units;
wherein the controller controls, among the plurality of power transmission antennas, a power transmission antenna from which a magnetic field is to be generated to place the power transmission antenna in a resonant state and controls, among the plurality of power transmission antennas, a power transmission antenna from which a magnetic field is not to be generated to place the power transmission antenna in a nonresonant state.

2. The wireless power feeding system according to claim 1, wherein the plurality of power transmission antennas include three power transmission antennas located so as to generate a magnetic field parallel to each axis of a predetermined three-dimensional Cartesian coordinate system.

3. The wireless power feeding system according to claim 1, further comprising a plurality of switches, each switches between applying and removing the AC voltage to the power transmission coil and/or the capacitor;
wherein the controller turns on and off the plurality of switches to control the resonant state of each of the plurality of power transmission antennas.

4. The wireless power feeding system according to claim 1, wherein the power transmission antenna includes a series resonance circuit in which the power transmission coil and the capacitor are connected in series.

5. The wireless power feeding system according to claim 1, wherein the power transmission antenna includes a parallel resonance circuit in which the power transmission coil and the capacitor are connected in parallel.

6. The wireless power feeding system according to claim 4, wherein the switch is connected in parallel with the capacitor.

7. The wireless power feeding system according to claim 5, wherein the switch is connected in parallel with the capacitor.

8. The wireless power feeding system according to claim 4, wherein the switch is connected in parallel with the power transmission coil.

9. The wireless power feeding system according to claim 4, wherein the switch is connected between the power transmission coil and the capacitor.

10. The wireless power feeding system according to claim 5, wherein the switch is connected between the power transmission coil and the capacitor.

11. The wireless power feeding system according to claim 4, wherein the switch is connected between the driving unit and the capacitor.

12. The wireless power feeding system according to claim 4, wherein the switch is connected between the power transmission coil and the driving unit.

13. The wireless power feeding system according to claim 1, placing the power transmission antenna in a resonant state and transmitting electric power to an in-vivo information acquiring device comprising a power receiving antenna including a power receiving coil for receiving electric power wirelessly transmitted.

14. The wireless power feeding system according to claim 13, wherein the in-vivo information acquiring device is a capsule endoscope.
